(19) United States
(12) Patent Application Publication
(10) Pub. No.: US 2011/0054595 A1
(43) Pub. Date: Mar. 3, 2011

(54) METHOD OF COATING MEDICAL DEVICES

(75) Inventors: Mark Landy, Atlanta, GA (US); Aleksey Tsvetkov, British Columbia (CA); Michael N. C. Chen, British Columbia (CA); Manus Tsui, British Columbia (CA)

(73) Assignee: MIV THERAPEUTICS, INC., Vancouver, British Columbia (CA)

(21) Appl. No.: 12/738,319

(22) PCT Filed: Oct. 20, 2008
(86) PCT No.: PCT/US08/80540

§ 371 Date: Nov. 2, 2010

(51) Int. Cl. A61F 2/82; B05D 7/00

(52) U.S. Cl. 623/1.39; 427/2.25

(57) ABSTRACT

Disclosed herein are medical devices, such as stents, comprising a porous substrate, such as a porous ceramic. Also disclosed herein are methods for impregnating the porous substrate with a composition comprising at least one lipid and at least one pharmaceutically active agent, where the porous substrate is microporous and/or nanoporous.

### RELATED APPLICATION

This application claims the benefit of priority under 35 U.S.C. §119(e) of U.S. Provisional Application No. 60/981,319, filed Oct. 19, 2007, the disclosure of which is incorporated herein by reference.

### FIELD OF THE INVENTION

Disclosed herein are coatings for medical devices, such as implantable medical devices (e.g., stents), and processes for making the same. Also disclosed are medical devices comprising a porous coating, and methods for impregnating the coating with a drug and lipid-containing composition either totally or partially. Methods for producing a smooth surface with drug evenly distributed along the length and diameter of the stent or device are also disclosed herein.

### BACKGROUND OF THE INVENTION

Medical devices, such as implantable medical devices are used in a wide range of applications including bone and dental replacements and materials, vascular grafts, shunts and stents, and implants designed solely for prolonged release of drugs. The devices may be made of metals, alloys, polymers or ceramics.

Arterial stents have been used for many years to prevent restenosis after balloon angioplasty (expanding) of arteries narrowed by atherosclerosis or other conditions. Restenosis involves inflammation and the migration and proliferation of smooth muscle cells of the arterial media (the middle layer of the vessel wall) into the intima (the inner layer of the vessel wall) and lumen of the newly expanded vessel. This migration and proliferation is called neointima formation. The inflammation is at least partly related to the presence of macrophages. The macrophages are also known to secrete cytokines and other agents that stimulate the abnormal migration and proliferation of smooth muscle cells. Stents reduce but do not eliminate restenosis.

Drug eluting stents have been developed to elute anti-proliferative drugs from a non-degradable polymer coating and are currently used to further reduce the incidence of restenosis. Examples of such stents are the Cypher® stent, which elutes sirolimus, and the Taxus® stent, which elutes paclitaxel. Recently it has been found that both of these stents, though effective at preventing restenosis, cause potentially fatal thromboses (clots) months or years after implantation. Late stent thrombosis is thought to be due to the persistence of the somewhat toxic drug or the polymer coating or both on the stent for long time periods. Examination of some of these stents removed from patients frequently shows no covering of the stent by the vascular endothelial cells of the vessel intima. This is consistent with the possible toxicity of the retained drugs or non-degradable polymer. The lack of endothelialization may contribute to clot formation.

There have been attempts to develop polymer-free coatings. However, these approaches have failed to produce the desired outcomes due to problems such as lack of mechanical integrity necessary to undergo device preparation and implantation, and may also result in undesirably fast release of the therapeutic agent.

Accordingly, there remains a need to develop new drug eluting stents having sufficient efficacy, mechanical integrity, and a surface that is biocompatible.

### SUMMARY OF THE INVENTION

One embodiment provides a stent, comprising at least one coating covering at least a portion of the device, the at least one coating comprising:

a porous substrate having a thickness and an average pore diameter of less than 1 μm; and

a composition impregnating at least 50% of the thickness of the porous substrate, the composition comprising at least one lipid and at least one pharmaceutically effective agent.

Another embodiment provides a method of coating a stent, comprising:

(a) providing a stent, at least a portion of the stent having a porous substrate having an average pore diameter less than 1 μm; and

(b) spraying the stent with a fluid composition comprising at least one pharmaceutically active agent and at least one lipid to impregnate at least some of the pores of the porous substrate with the composition;

(c) spraying the stent with a solvent; and

(d) repeating steps (b) and (c) at least once, e.g., at least twice.

Another embodiment provides method of coating a medical device (e.g., a stent) comprising:

providing a medical device/stent, at least a portion of the device/stent having a porous substrate having an average pore diameter less than 1 μm; and

dipping the medical device/stent in a composition comprising at least one lipid and at least one pharmaceutically active agent to impregnate at least some of the pores of the porous substrate with the composition

spinning the device to remove excess composition.

In one embodiment, the method further comprises spraying the device with either a solvent or a dilute solution comprising the composition.

Another embodiment provides a method of coating a medical device comprising:

providing a medical device, at least a portion of the device having been previously coated with a porous substrate having an average pore diameter less than 1 μm;

subjecting the device to a vacuum; and

maintaining the vacuum while applying to the device a composition comprising at least one lipid and at least one pharmaceutically active agent to impregnate at least some of the pores of the porous substrate with the composition.

In one embodiment, the vacuum is maintained at −20 mm Hg or greater.

Another embodiment provides a method of coating a medical device comprising:

providing a medical device, at least a portion of the device having been previously coated with a porous substrate having an average pore diameter less than 1 μm; and

dipping the medical device in a composition comprising at least one lipid and at least one pharmaceutically active agent to impregnate at least some of the pores of the porous substrate with the composition;

spraying the device with either a solvent or a dilute solution of the composition.

### BRIEF DESCRIPTION OF THE DRAWINGS

FIG. 1 is a flowchart illustrating possible coating methods herein;

FIG. 2 is a schematic of a device coated with a porous substrate impregnated with a composition comprising at least one lipid and at least one pharmaceutically active agent;

FIG. 3 schematically depicts the use of a spray technique to refinish a coating;

FIG. 4 schematically depicts the use of a wetting step prior to coating the porous substrate with a lipid/drug composition;

FIG. 5A is a photograph of a stent coated by the method of Example 3;

FIG. 5B is a photograph of a stent coated by the method of Example 4;

FIG. 6A is a photograph of a stent exposed to the composition of Example 2 without a pre-spraying step;

FIG. 6B is a photograph of a stent coated by the method of Example 6;

FIG. 7 schematically depicts a vacuum chamber apparatus for coating a stent exposed to the composition of Example 2;

FIG. 8 schematically depicts a vacuum chamber apparatus for spraying a stent with the composition of Example 2;

FIG. 9 is a graph showing the amount of drug released (y-axis) over time (x-axis) from 19 mm stents coated with different batches of the composition of Example 2 by the method of Example 10;

FIG. 10 is a graph showing the amount of drug released (y-axis) over time (x-axis) from 29 mm stents coated with different batches of the composition of Example 2 by the method of Example 10;

FIG. 11 is a graph showing the amount of drug released (y-axis) over time (x-axis) for stents subjected to the multiple spray process of Example 10 compared with stents subjected to the dip/spin process of Example 3; and

FIG. 12 is a graph showing the amount of drug released (y-axis) over time (x-axis) for two different stent designs coated by the method of Example 10.

### DETAILED DESCRIPTION

There have been efforts within the medical device industry to develop a stent that elutes drugs in a controlled manner. Stents coated with a drug alone had exhibited a burst release of drug upon exposure to the bloodstream. Polymer-coated stents were then generated to contain the drugs, thereby reducing the release rate by requiring the drug to diffuse through the polymer film. However, such stents suffered the long-term thromboses problems mentioned previously. Moreover, because the release rate of drugs from prior art polymer coatings depended substantially on the rate of diffusion of the drug through the polymer coating, the diffusion rate may be too slow to deliver the desired amount of drug to the body over a desired time. As a result, a significant amount of the drug may remain in the polymer coating.

Accordingly, one embodiment provides a medical device comprising a porous substrate where the porosity volume and average pore size are designed to provide tortuous pathways for the drug to be released from the coating. The rate of drug release from a porous substrate is increased compared to the rate of release from a polymer coating, which relies primarily on diffusion. In one embodiment, the average pore diameter is less than 1 μm.

In another embodiment, the drug impregnates the porous substrate in the presence of at least one lipid. In certain embodiments, the porous structure may not be sufficient to decrease the release rate of drugs from the substrate to a desired level. A lipid-containing composition can decrease the rate of diffusion of the drug from the pores, as the drug must diffuse through the lipid as well as through the porous network. Moreover, the at least one lipid can help the drug adhere to the stent.

Generally, porous substrates can have an interconnecting 3-dimensional structure capable of containing organic or inorganic materials within the pores. The materials may be present for chemical and/or mechanical reinforcement, or even for later release during application (e.g., a drug delivery system), and even as a sacrificial layer/coating for protection. Typically, these materials are infiltrated within the pores via various processing routes, including vapor deposition (chemical or physical), impregnation (external or natural capillary forced), dipping, spinning, and infiltration via various spraying techniques available in the market.

Normal coating processing techniques, such as dipping and spray coating a solution containing the drug, may be generally ineffective for impregnating microporous or nanoporous substrates with a composition comprising a drug and at least one lipid. For example, it has been discovered that conventional spray coating processes with a solution containing the drug and lipid can result in precipitation of drug particles measuring up to a few microns in diameter. These particles may block the pore openings and may hamper the ingress of a lipid/drug composition into the porous substrate. Other factors that may hamper coating uniformity include solvent evaporation that, with possible precipitation of the solute, may create rough/patchy surface finish along with the possibilities of heterogeneous concentration within and along the 3-D structure.

Disclosed herein are methods for preparing new device coatings in which an appreciable amount of the pores are impregnated with a drug and have a sufficiently uniform drug distribution. These methods allow production of new devices. Accordingly, one embodiment provides a medical device, comprising at least one coating covering at least a portion of the device, the at least one coating comprising:

a porous substrate having a thickness and an average pore diameter of less than 1 μm; and

a composition impregnating at least 50% of the thickness of the porous substrate, the composition comprising at least one lipid and at least one pharmaceutically effective agent.

Such a device has not been prepared by conventional methods.

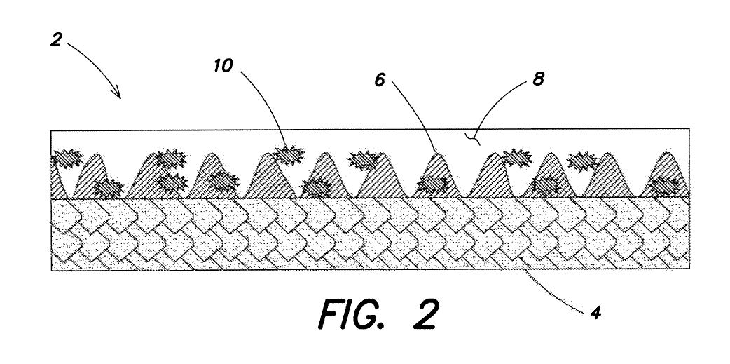

FIG. 2 schematically depicts an embodiment of the coated devices disclosed herein. “Coated medical device” as used herein includes those devices having one or more coatings, i.e., at least one coating. The at least one coating can comprise one coating covering at least a portion of the device, e.g., all or some of the device. For example, where the device is a stent, the coating can cover the entire stent, or can cover only the portion of the stent that contacts a body lumen. The device may employ more than one coating for different portions of the device, or can employ multiple layers of coatings.

A section of device 2 comprises surface 4 coated with a porous substrate 6, the surface of which is schematically depicted. Impregnating substrate 6 is a composition comprising one or more lipids 8, which acts as a vehicle for pharmaceutically active agent 10. The agent 10 may contact the porous substrate 6, or may be suspended in the lipid(s) 8 without contacting substrate 6. The agent 10 may be embedded in the lipid(s) 8 in molecular or particulate form.

In one embodiment, the composition has sufficient flowability to impregnate the porous substrate to at least 50% of the thickness as measured from the top surface of the porous substrate (that does not contact the device). In another embodiment, the composition impregnates the porous substrate to at least 60%, at least 70%, or at least 80% the thickness of the porous substrate, or even at least 90% the thickness of the porous substrate. In one embodiment, the flowability can be achieved by supplementing the formulation with a spraying step with either a solvent or a more dilute solution containing the formulation to improve the coating penetration, uniformity, and/or increase the drug loading.

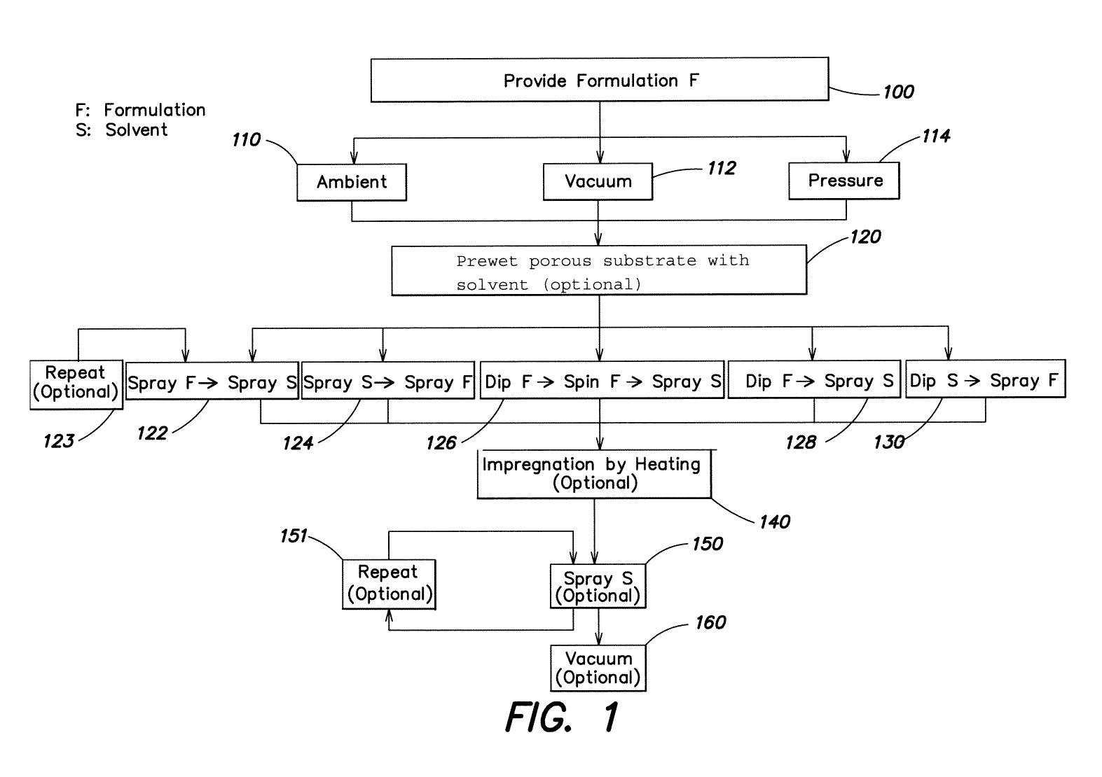

FIG. 1 is a flowchart showing the possible methods of coating a porous substrate with the composition comprising at least one lipid and at least one pharmaceutically active agent to allow penetration of at least 50% the thickness of the porous substrate. FIG. 1 schematically depicts a step 100 of providing a Formulation F, which comprises at least one pharmaceutically active agent and at least one lipid. This stent can be coated with the formulation under ambient conditions 110, under vacuum 112, or under pressure 114. Formulation F can optionally contain a solvent to achieve a homogeneous solution comprising the lipid(s) and agent(s).

One embodiment provides an optional prewetting process 120 to initially coat the stent with a solvent prior to adding the formulation F.

One embodiment of coating the stent provides a spray process 122 involving alternating sprays of the formulation F with solvent S. The formulation F is initially sprayed onto the porous substrate of the stent followed by a spray of solvent S. This spray process can be repeated at step 123 at least once, or at least twice to increase the extent of penetration of formulation F into the porous substrate. Another embodiment provides a spray process 124 involving alternating sprays of the solvent S with formulation F. Process 124 can also be repeated at least once or at least twice (not shown in FIG. 1).

Another embodiment provides methods of dipping the porous substrate of the stent in formulation F, followed by additional processes. For example, process 126 provides a dipping step where the stent is dipped into the formulation F, followed by a spinning step to remove excess formulation and/or to provide a more uniform coating. A spray S of solvent can further redistribute the formulation and increase the coating uniformity, and/or increase the extent of penetration of the formulation into the porous substrate. In another embodiment, process 128 eliminates the spinning step of process 126, whereas a solvent spray S is applied directly after dipping the porous substrate in the formulation F. In yet another embodiment, process 130 involves dipping the porous substrate into a solvent S followed by spraying the substrate with formulation F.

The selection of these various coating processes can depend on the type of lipid and the drug and relative solubilities in a solvent, the pore size, etc.

In one embodiment where the drug/lipid composition possess heat stability and the viscosity of the composition decreases with temperature, the method comprises heating the device at step 140 at a temperature that reduces the viscosity of the drug formulation to a sufficiently low value, allowing the formulation to flow and further impregnate or penetrate the porous substrate.

In another embodiment, a final spray step 150 with solvent S can be performed to increase coating uniformity of the formulation F and/or penetration into the porous substrate due to its reduced viscosity or reduced surface tension. The spray step can be repeated at least once or twice at step 151.

In yet another embodiment, a vacuum step 160 can be performed to cause the lipid/drug composition to flow into the pores.

Steps 140, 150, and 160 are optional as the prior steps may have resulted in a sufficiently uniform coating that penetrates the porous substrate to a sufficient depth, e.g., at least 50% of the thickness of the porous substrate. Thus, coating steps 122-130 can be performed without additional steps 140, 150, and 160, or succeeded by one, two, or all of steps 140, 150, and 160. In other embodiments not shown in FIG. 1, the order of steps 140, 150, and 160 can be changed, e.g., the vacuum step 150 can precede a final spray step 150, or a heating step 140 can succeed the spray step 150. Both a vacuum step 150 and a heating step 160 can be applied with or without a spray step 150 (and repeated spray 151).

In one embodiment, the processing involves a final drying step to remove the solvent. The drying can be achieved by exposing the coated stent to at least one of a vacuum, heat, and/or ambient/room temperature conditions for a period of time sufficient to remove substantially all of the solvent.

It has been discovered that applying a formulation containing the at least one lipid and drug, even in dilute solution form, by conventional methods can often result in one or more of a nonuniform distribution of the formulation throughout the stent, incomplete impregnation of the pores due to the viscosity of the lipid/drug-containing formulation, crystallization or precipitation of the drug thereby blocking the pores of the substrate, or webbing between the pores. In one embodiment, the methods comprise at least one solvent spray step succeeding and/or preceding the application of a formulation on the porous substrate.

One embodiment provides a method of coating a medical device, such as a stent, comprising:

providing a medical device, such as a stent, at least a portion of the device/stent having a porous substrate having an average pore diameter less than 1 μm; and

exposing the device/stent to a fluid composition comprising at least one pharmaceutically active agent to impregnate at least some of the pores of the porous substrate with the composition.

In one embodiment, the fluid composition further comprises at least one lipid. In another embodiment, the fluid composition further comprises at least one solvent. The step of exposing the device/stent can comprise dipping or spraying the device with the fluid composition. In another embodiment, the method further comprises spraying the exposed device/stent with a solvent. In one embodiment, the exposing step can be alternated with the spraying step. Accordingly, one embodiment provides a method of coating a stent, comprising:

(a) providing a stent, at least a portion of the stent having a porous substrate having an average pore diameter less than 1 μm; and

(b) spraying the stent with a fluid composition comprising at least one pharmaceutically active agent and at least one lipid to impregnate at least some of the pores of the porous substrate with the composition;

(c) spraying the stent with a solvent; and

(d) repeating steps (b) and (c) at least once, e.g., at least twice.

In one embodiment, the spraying in (b) is preceded by an initial spray of solvent. In one embodiment, the fluid composition further comprises a solvent. The solvent in the fluid composition can be the same as or be miscible with the solvent in one or more of the initial solvent spray that precedes (b), and the spray of (c).

The solvent can be any of a number of low viscosity and/or low surface tension solvents. In one embodiment, the solvents are capable of quickly dissolving the drug and at least one lipid. In one embodiment, the solvents are chosen from ethyl alcohol, acetone, DMSO, methyl alcohol, and mixtures thereof.

In one embodiment, the at least one pharmaceutically active agent is soluble in the solvent. Without wishing to be bound by any theory, the solvent spray (e.g., in one or more of steps 120, 122, 123, 124, 126, 128, and 150) can achieve one or more of the following functions: (1) reduce the viscosity and/or surface tension of the formulation; (2) dissolve or redissolve precipitates or particles that had crystallized from the formulation, e.g., drug precipitates or particles; (3) assist in penetration of the formulation into the pores by capillary action, where one or more of (1)-(3) can result in a greater extent of penetration of the formulation into the porous substrate (e.g., greater than 50% of the thickness of the substrate). The spraying can flood the substrate with solvent, or even a dilute solution (e.g., less than 50%, less than 25%, less than 10%, less than 5%, or even less than 1% the concentration of the fluid composition of step (b)). Upon evaporation of the solvent, a finished layer may result.

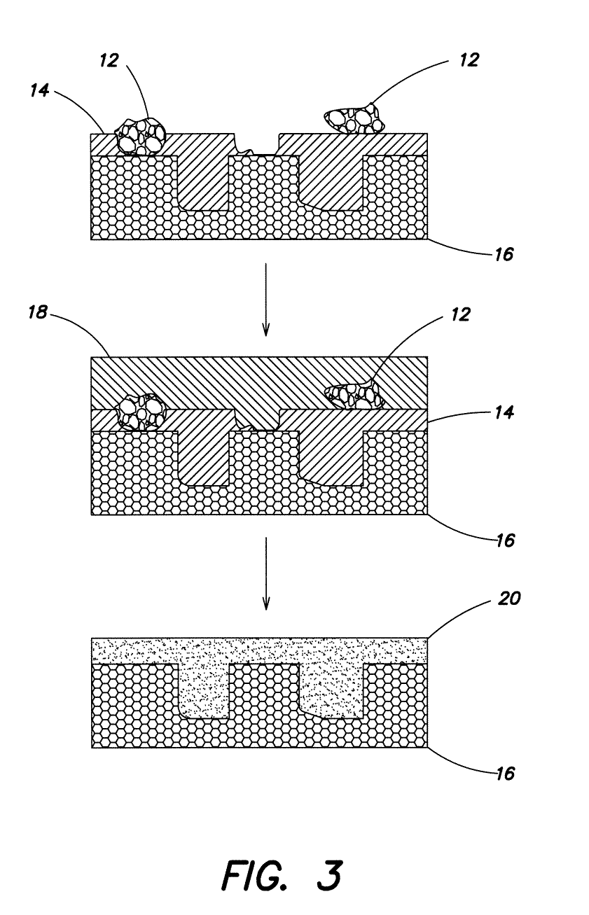

This process is schematically illustrated in FIG. 3, which shows one embodiment of a porous substrate 16 after the spraying of the fluid composition 14 of step (a). In this embodiment, spraying the fluid composition directly onto the substrate resulted in drug precipitates or crystals 12 that can disrupt the uniformity of the coating and prevent the drug from entering the pores. Spraying a solvent 18 can dissolve or redissolve the precipitates 12, resulting in a uniform solution 20 that can now cause more of the drug to penetrate the pores of substrate 16.

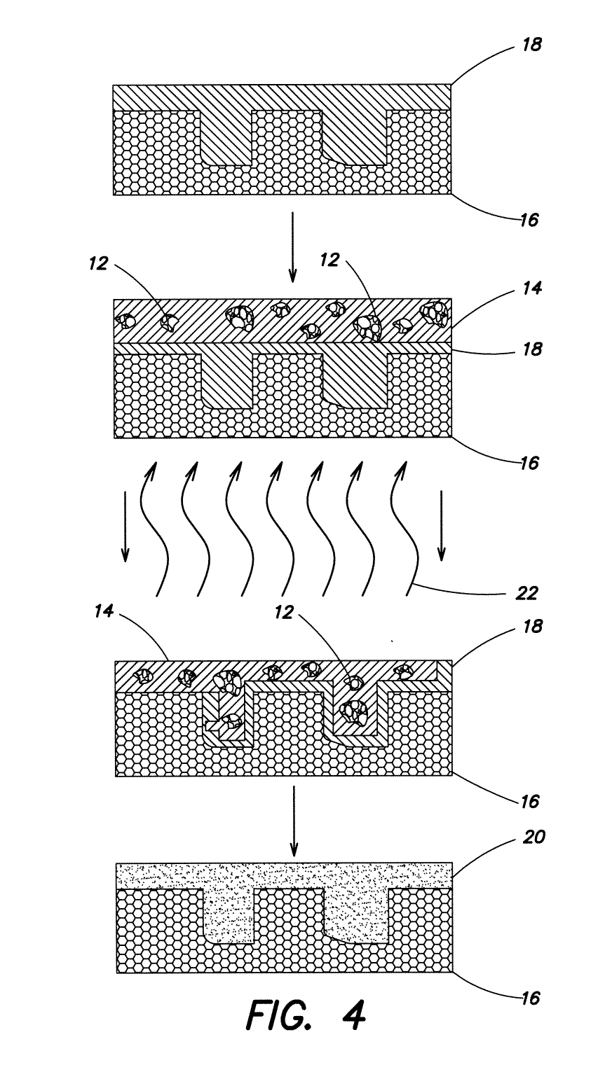

Another possible method to reduce the viscosity and/or surface tension of the coating and therefore increase its penetration into the pores involves initially spraying the porous substrate with a solvent. The composition comprising the at least one pharmaceutically active agent and at least one fluid is then sprayed on the solvent layer. The first layer of sprayed solvent on the surface of the porous substrate can increase the flowability of the composition and allow it to penetrate deep into the pores. This method is schematically depicted in FIG. 4, where porous substrate 16 is initially flooded by spraying with a solvent 18. In certain instances, a portion of the at least one pharmaceutically active agent has precipitated out of fluid composition 14 containing at least one lipid. The presence of solvent 18 can reduce the viscosity of composition 14 and/or redissolve the precipitates and/or effect capillary action to improve penetration of composition 14, resulting in a more uniform coating 20.

Other coating methods besides the above spraying processes can be used to achieve improved penetration of a drug/lipid formulation. One embodiment provides a method of coating a medical device comprising:

providing a medical device, at least a portion of the device having been previously coated with a porous substrate having an average pore diameter less than 1 μm; and

dipping the medical device in a composition comprising at least one lipid and at least one pharmaceutically active agent to impregnate at least some of the pores of the porous substrate with the composition; and

spinning the device to remove excess composition.

In one embodiment, the dipping comprises immersing the medical device in the formulation for a period of time to thoroughly coat the surface. In one embodiment, the immersing occurs over a time period ranging from 1 s to 1 day, such as a time period ranging from 1 s to 300 s.

In one embodiment, the spinning comprises spinning the device at a rate of 30-10000 rpm to remove the excess composition from the surface.

In certain embodiments, the coating after spinning may be sufficiently uniform for use. In other embodiments, the coating produced after spinning may not be sufficiently uniform in appearance. In this situation, the method further comprises spraying the device (after dipping and spinning) with a solvent or a dilute solution comprising the composition, such that the solvent or dilute solution is capable of fully or partially dissolving the drug composition. In one embodiment, the spraying occurs while the device is rotated, e.g., at a speed of 10-500 rpm. The spraying may decrease the viscosity of the coating and would allow the coating to spread across the device to create a more uniform coating. If a dilute solution is used, it can be diluted from 1-50 times the dilution of the composition. Using the diluted formulation instead of the solvent alone may allow the addition of an extra amount of the composition where a higher loading of the pharmaceutically active agent is desired.

The spraying with a solvent or dilute solution may also redissolve precipitates or particles that have formed during the dipping and/or spinning processes, as discussed above.

Another embodiment provides a method of coating a medical device comprising:

providing a medical device, at least a portion of the device having been previously coated with a porous substrate having an average pore diameter of less than 1 μm;

wetting the porous substrate with at least one solvent; and

dipping the medical device in a composition comprising at least one lipid and at least one pharmaceutically active agent to impregnate at least 50% of a thickness of the porous substrate.

In one embodiment, this method reduces the viscosity of the composition on the surface of the medical device (stent), thus increasing the flowability of the composition into the pores.

In another embodiment where the drug/lipid composition possess heat stability and the viscosity of the composition decreases with temperature, the method comprises dipping the medical device in the composition and heating the device at a temperature that reduces the viscosity of the drug formulation to a sufficiently low value, allowing the formulation to flow and impregnate the porous substrate.

In addition or in the alternative of reducing the viscosity of the coating to improve the flowability, the rate of flow of the lipid/drug composition into the pores can be improved by subjecting the device to a vacuum and applying the composition to the device. Accordingly, another embodiment provides a method of coating a medical device comprising:

providing a medical device, at least a portion of the device having been previously coated with a porous substrate having an average pore diameter less than 1 μm;

subjecting the device to a vacuum; and

maintaining the vacuum while applying to the device a composition comprising at least one lipid and at least one pharmaceutically active agent to impregnate at least some of the pores of the porous substrate with the composition.

The vacuum applying steps can occur simultaneously or sequentially. For example, the device can be subjected to a vacuum followed by dipping or spraying. The device can be subjected to a vacuum for a time period ranging from 1 s to 1 hour, such as a time period of 1-300 s. In one embodiment, after the dipping or spraying, the device is spun to remove any excess composition.

In one embodiment the stent can be initially sprayed with a solvent or a solvent mixture with a low evaporation rate (0.02 to 3), sprayed with the composition and then placed under vacuum, e.g., immediately after spraying. The underlying solvent layer can dissolve the composition thereby reducing its viscosity while the vacuum aids in causing penetration of the composition further into the pores. The vacuum can be released and then resumed in intervals in order to improve the penetration of the formulation.

Another embodiment provides a method of coating a medical device comprising:

providing a medical device, at least a portion of the device having been previously coated with a porous substrate having an average pore diameter less than 1 μm; and

dipping the medical device in a composition comprising at least one lipid and at least one pharmaceutically active agent to impregnate at least some of the pores of the porous substrate with the composition

spraying the device with either a solvent or a dilute solution of the composition.

In one embodiment, the device can be immersed in the composition for a period of 1-300 seconds. After removal of the device from the composition, the device can be sprayed with a solvent or a dilute solution comprising the composition while the stent is being rotated, e.g., at a rate of 1-3000 rpm.

Another embodiment provides a method of coating a medical device comprising:

providing a medical device, at least a portion of the device having been previously coated with a porous substrate having an average pore diameter less than 1 μm; and

exposing the device to a composition comprising at least one pharmaceutically active agent and at least one solvent to impregnate at least some of the pores of the porous substrate with the composition.

In this embodiment, a solution can be formed by dissolving the pharmaceutically active agent in one or more solvents. In another embodiment, an emulsion can be formed comprising water and one or more immiscible solvents, the emulsion further comprising the agent(s). Optionally, the emulsion can include at least one lipid in the situation where a lipid-containing vehicle is desired in the coating to contain the drug. The emulsion can further contain surfactants to stabilize the emulsion. One of ordinary skill in the art can select appropriate surfactants, depending on the solvent and drug types to achieve a stable emulsion.

In one embodiment, the porous substrate can have pores and voids sufficiently large enough to contain a drug yet have passageways that permit a drug to release from the pores of the substrate and enter the aqueous solution. The substrate can thus act as a drug reservoir and the porosity properties, e.g., porosity volume and/or pore diameter, can dictate the release rate of the drug from the substrate. In one embodiment, the substrate has a porosity volume ranging from 30-70% and an average pore diameter ranging from 0.3 μm to 0.6 μm. In one embodiment, the porous substrate has a porosity volume ranging from 30 to 70% and an average pore diameter ranging from 0.3 μm to 0.6 μm. In other embodiments, the porosity volume ranges from 30 to 60%, from 40 to 60%, from 30 to 50%, or from 40 to 50%, or even a porosity volume of 50%. In yet another embodiment, the average pore diameter ranges from 0.4 to 0.6 μm, from 0.3 to 0.5 μm, from 0.4 to 0.5 μm, or the average pore diameter can be 0.5 μm.

A porous substrate may offer an opportunity for a single drug type to exhibit dual functionality. In conjunction with a drug impregnating the porous substrate, a film comprising a lipid bilayer and at least one pharmaceutically active agent can coat the substrate.

In one embodiment, the porous substrate forms the surface of the entire layer of the stent. In another embodiment, only certain sections of the stent have the porous substrate, e.g., only the abluminal side of the stent has the porous substrate. Different parts of the stent can be coated with different lipids in combination with different drugs, e.g., one drug type on the ends of the stent, a different drug type on the outer surface, and a different drug on the inner surface.

In one embodiment, the porous substrate is present in the stent itself. For example, the stent surface can contain isolated pores, or a series of interconnecting pores. In another embodiment, the porous substrate comprises a material that was deposited on the stent surface. In one embodiment, a porous ceramic is deposited on the stent surface.

In one embodiment, the porous substrate can be a ceramic, such as any ceramic known in the art to be biocompatible, e.g., metal oxides such as titanium oxide, aluminum oxide, and indium oxide, metal carbides such as silicon carbide, and one or more calcium phosphates such as hydroxyapatite, octacalcium phosphate, α- and β-tricalcium phosphates, amorphous calcium phosphate, dicalcium phosphate, calcium deficient hydroxyapatite, and tetracalcium phosphate.

In one embodiment, the substrate is a calcium phosphate coating, such as hydroxyapatite. The calcium phosphate coating may be deposited by electrochemical deposition (ECD) or electrophoretic deposition (EPD). In another embodiment the coating may be deposited by a sol gel (SG) or an aero-sol gel (ASG) process. In another embodiment the coating may be deposited by a biomimetic (BM) process. In another embodiment the coating may be deposited by a calcium phosphate cement (CPC) process.

In one embodiment, the porous substrate can comprise a steel mesh or a polymer.

In one embodiment, the porous substrate has a thickness of 10 μm or less. In other embodiments, e.g., where the device is an orthopedic implant, the porous substrate can have a thickness ranging from 10 μm to 5 mm, such as a thickness ranging from 100 μm to 1 mm.

In another embodiment, the device is a stent, and the thickness of the substrate is selected to provide a sufficiently flexible coating that stays adhered to the stent even during mounting and expansion of the stent. A typical mounting process involves crimping the mesh-like stent onto a balloon of a catheter, thereby reducing its diameter by 75%, 65%, or even 50% of its original diameter. When the balloon mounted stent is expanded to place the stent adjacent a wall of a body lumen, e.g., an arterial lumen wall, the stent, in the case of stainless steel, can expand to up to twice or even three times its crimped diameter. For example, a stent having an original diameter of 1.7 mm can be crimped to a reduced diameter of 1.0 mm. The stent can then be expanded from the crimped diameter of 1.0 mm to 3.0 mm. Accordingly, in one embodiment, the substrate has a thickness of no more than 2 μm, such as a thickness of no more than 1 μm.

In one embodiment, the substrate is well bonded to the stent surface and neither forms significant cracks nor flakes off the stent during mounting on a balloon catheter and placement in an artery by expansion. In one embodiment, a coating that does not form significant cracks can have still present minor crack formation so long as it measures less than 300 nm, such as cracks less than 200 nm, or even less than 100 nm.

The pharmaceutically active agent(s) in the porous substrate can be hydrophilic, hydrophobic, or amphipathic. In one embodiment the agent impregnating the porous substrate is soluble in the pliable vehicle. In another embodiment the agent is insoluble in the vehicle.

In one embodiment, the drug is distributed uniformly throughout the stent surface, e.g., there is a uniform concentration of drug. In one embodiment, a uniform concentration can be determined by cutting sections of the stent of equal width (e.g., cut the stent in four equal section) in a direction perpendicular to the longitudinal axis. In one embodiment, the drug concentration of drug in each section does not vary by more than ±5%, or does not vary by more than ±3%.

In another embodiment, a uniform concentration is determined by the variation in the amount of drug from stent to stent. In one embodiment, the variation is within 10% of a target specification, e.g., no more than 7%, or no more than 5%. The target specification can be a desired amount of drug eluted from the stent and the amount of drug loaded on the stent.

### EXAMPLES

### Example 1

This Example describes the use of hydroxyapatite-coated stents as prepared in U.S. Provisional Application No. 60/978,988, filed Oct. 10, 2007, U.S. application Ser. No. 12/060,604, filed Apr. 1, 2008, and in Tsui, Manus Pui-Hung, “Calcium Phosphate Coatings on Coronary Stents by Electrochemical Deposition,” M.A.Sc. diss., University of British Columbia, University, 2006, the disclosures of which are incorporated herein by reference.

The hydroxyapatite coating uniformly covered the stent and the thickness is ˜0.5 um. An expansion test was performed after the ECD-HAp coated stent was air dried. An Encore™ 26 INFLATION DEVICE KIT was used to inflate the catheter to 170 psi. The expanded stent was observed under SEM. No separation of the coating was visible even in the areas of the highest strain due to the expansion for magnifications up to 10,000×. The stent strain was accommodated by the coating through nano-size localized cracking, not visible under the microscope.

### Example 2

This Example describes the preparation of a composition comprising sirolimus as the pharmaceutically active agent and castor oil as the lipid.

Castor oil (1000 mg) was added to 9000 mg of ethanol and mixed to give a clear solution. Sirolimus (100 mg) was added to 660 mg of the above solution and mixed. 2.0 g of ethanol was then added to the sirolimus/castor oil mixture and stirred to give a clear solution.

### Example 3

This Example describes the coating of the hydroxyapatite (HAp) coated stent of Example 1 with the composition of Example 2 by dipping and spin coating.

The HAp coated stent was immersed in the composition of Example 2 for a period of 60 seconds. The stent was then withdrawn from the formulation and the excess liquid on the surface was removed by placing the stent on a stent holder connected to a rotating device. The stent was rotated about its longitudinal axis with a rotation speed of 5000 rpm for a period of 10 seconds.

### Example 4

This Example describes the spraying the resulting coated stent of Example 3 with the solvent to achieve a surface finish.

The stent of Example 3 was sprayed with ethanol or a diluted version of the formulation prepared in Example 2, e.g. 40 times dilution in ethanol, using a spraying machine (e.g., a MicroMist spraying machine). A wet film forms on the surface of the stent dissolving and redistributing any precipitates from previous processing steps, thereby improving the uniformity of the coating. The stent is further placed under vacuum (−30 mm Hg) for 12 hours to remove residual solvents.

### Example 5

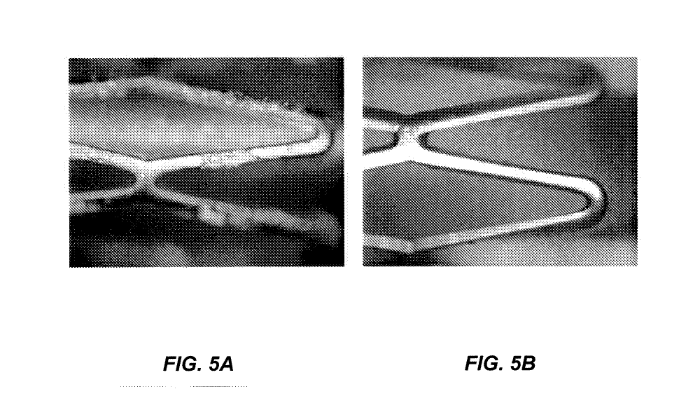

An optical picture of the stent prepared using Examples 3 and 4 are shown in FIGS. 5A and 5B, respectively. Although the dip and spin coated stents of Example 3 may be suitable for some uses, the surface finish of Example 4 provides the stent with a more uniform look in appearance. It is observed that by spraying the surface of the previously deposited structure, the effect of diffusion into the pores were able to redistribute the solute evenly throughout the surface. The spraying created a large quantity of liquid on the surface, hence enabling a slower drying. More optimal results were observed when a very dilute amount of solute/solids <0.2% was present in the sprayed solvent.

### Example 6

The HAp coated stent of Example 1 was sprayed with ethanol as the first step. Before the ethanol dried, the stent was immediately sprayed with the composition prepared as in Example 2.

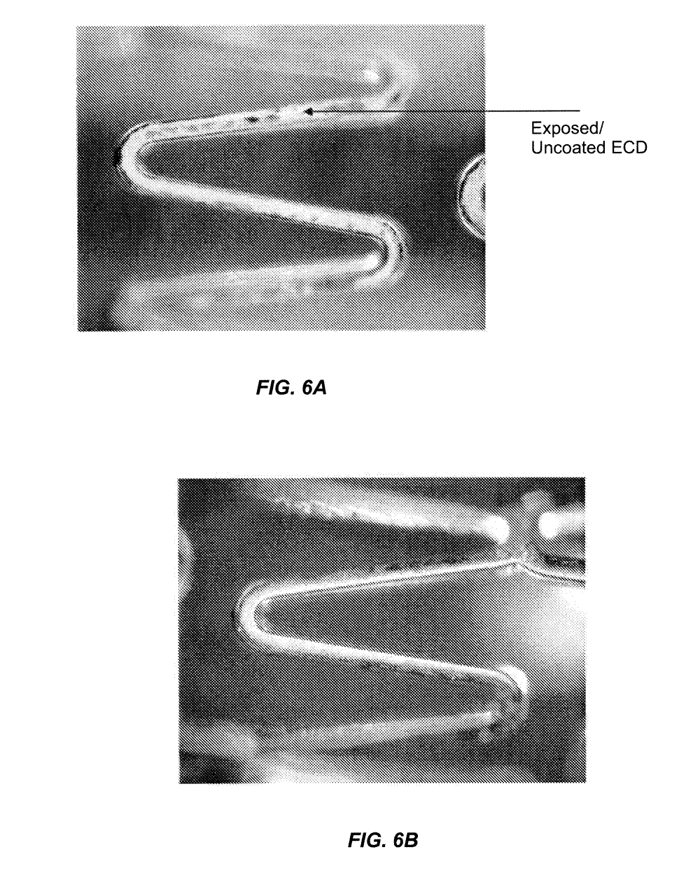

FIGS. 6A and 6B are optical images of a stent exposed to the composition of Example 2 without pre-spraying the surface with solvent (FIG. 6A) versus a coating that was pre-sprayed according to the present Example. In FIG. 6B, the porous HAp coating is shown to be completely covered by the lipid/drug composition and evenly distributed along the surface of the entire stent. This may be explained by fact that ethanol has a low wetting angle, allowing it spread entirely on the surface and penetrate into pores to create a wet surface. Once the composition of Example 2 was sprayed, the effect of diffusion, and/or capillary action may result in redistribution of the solute evenly into the pores and homogeneously throughout the surface as the liquids mixed. Optimal results were observed when the entire stent surface was sprayed with a thick layer of solvent observable by the naked eye where no dripping/droplets were observed.

### Example 7

This Example describes a vacuum-assisted dip-coating method.

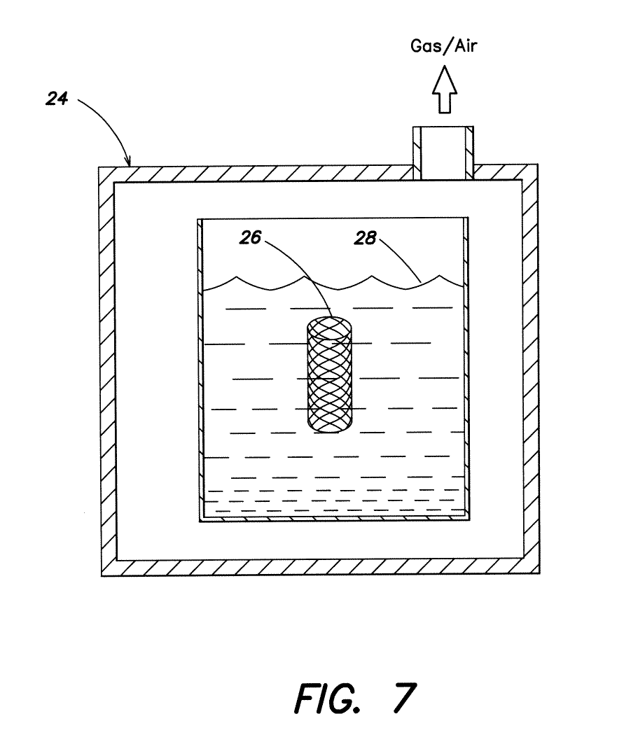

The HAp coated stent of Example 1 was placed in a vacuum chamber. A schematic of the vacuum chamber/spray apparatus 24 is schematically depicted in FIG. 7. The stent 26 was placed in a flask (not shown) in the chamber 24 and the composition of Example 2 was placed in a vessel isolated from the stent.

The air/gas initially in the chamber was evacuated until the vacuum in the chamber reached the pressure of −22 mm Hg. The composition 28 was then slowly released into the flask containing the stent until it completely submerged the porous HAp, at which point the pressure was maintained at −22 mm Hg for 10 seconds. If necessary, further processing techniques can be applied, e.g., spinning or the spraying method of Example 4, to improve the quality of the coating.

### Example 8

This Example describes an alternative vacuum-assisted dip-coating method.

Using the vacuum chamber 24 of Example 7, the porous HAp coated stent of Example 1 was immersed in a flask containing the composition of Example 2. The flask was then placed under vacuum at the target pressure of ≧−22 mm Hg and maintained at the level for 30 seconds, after which time the stent was removed from the solution. If necessary, further processing techniques can be applied, e.g., spinning or the spraying method of Example 4, to improve the quality of the coating.

### Example 9

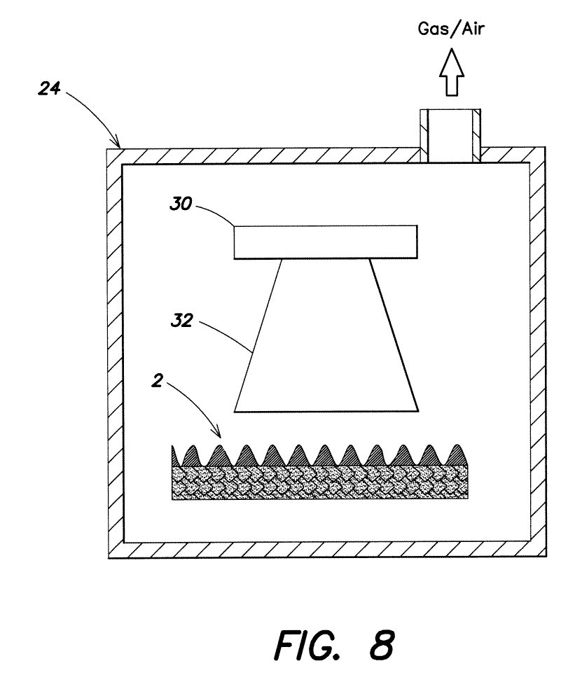

This Example describes a method for spray coating a porous substrate, as in Example 4, under vacuum conditions. A schematic of the vacuum chamber/spray apparatus 24 is schematically depicted in FIG. 8.

The porous HAp coated stent 2 of Example 1 was placed in a vacuum chamber and subjected to a ≧−30 mm Hg vacuum. Upon achieving this pressure, the composition 32 of Example 2 was sprayed via sprayer 30 onto the surface of the porous HAp coated stent 2 to flood the surface with sufficient formulation where no dripping/droplets are observed. The stent was maintained at this negative pressure for 1 minute before the pressure was released and the sample removed from the vacuum chamber. The coating was then allowed to dry in a desiccator at room temperature for 12 hours.

### Example 10

This Example describes a multi-step spraying process, including solvent spraying steps that can further liquefy a composition comprising at least one drug and at least one lipid. This solvent spraying process in turn can result in a lower viscosity and low surface tension liquid allowing it flow much more freely on the surface of substrate.

The HAp coated stent of Example 1 was sprayed with ethanol as the first step. Before the ethanol dried, the stent was immediately sprayed with the composition prepared as in Example 2.

A high volume spray of micron sized droplets of ethanol is directed at the stent via a stationary nozzle to double the volume of solvent reaching the surface. The stent substrate traveled horizontally at rate of 0.1 in/sec and maintaining constant rotational speed (120 rpm) throughout the process. A homogeneous wet surface results with sufficient volume to dissolve the composition of Example 2 and increase penetration into the porous substrate porosity. Care is taken to ensure that an excess amount of solvent is not applied to cause the liquefied coating to drip, sag, or streak while being spun.

The multiple spray process of formulation followed by ethanol spray was repeated twice.

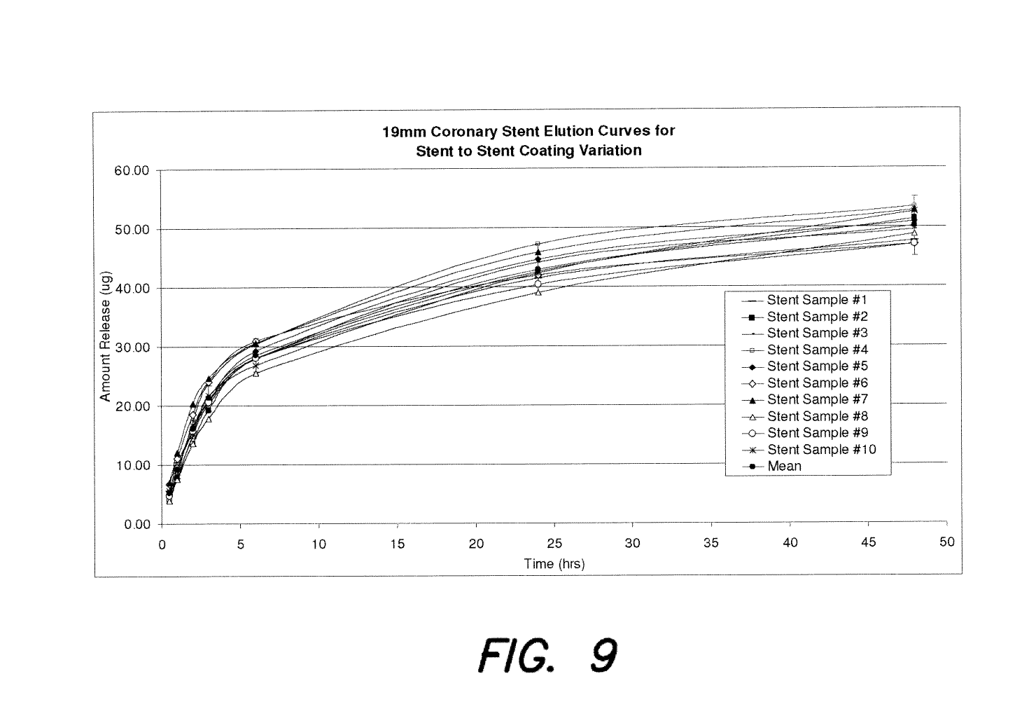

The amount of drug in the stent was evaluated by determining its release over time as monitored high-performance liquid chromatography (HPLC). FIG. 9 is a graph showing the amount of drug released (y-axis) over a time period (x-axis) of approximately 50 hours for 10 stents (19 mm) coated with different batches of the composition of Example 2 subjected to the present multiple-spray process. It can be seen that the amount of drug released is substantially uniform with minimal variation from stent to stent.

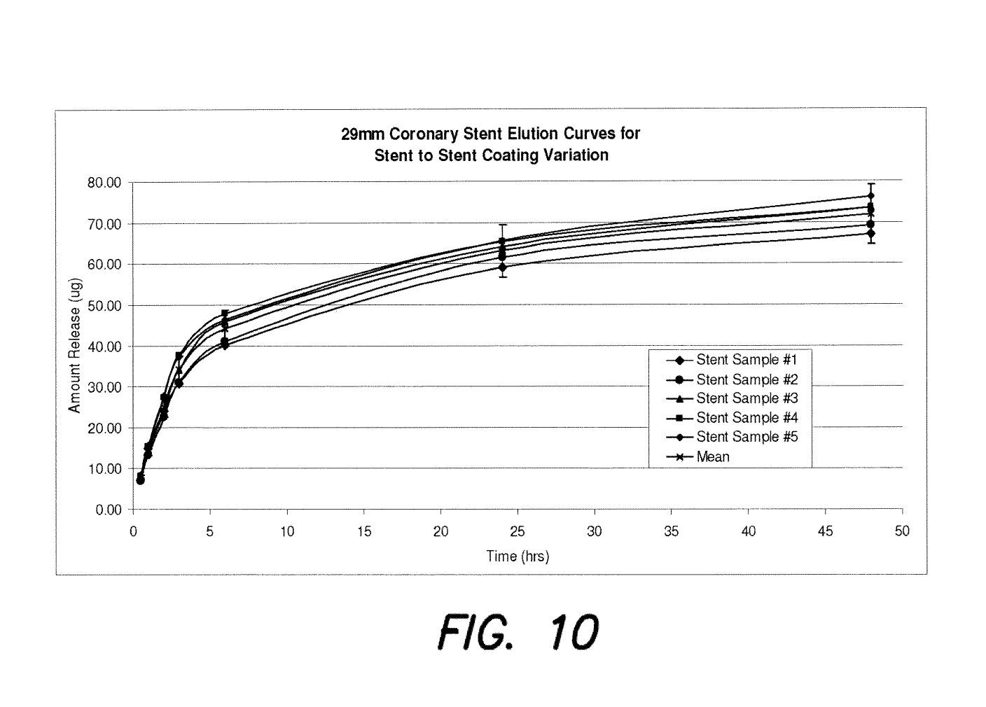

FIG. 10 is a graph resulting from an experiment similar to that of FIG. 9. Five 29 mm stents were subjected to different batches of the composition of Example 2 subjected to the multiple-spray process. Again, It can be seen that the amount of drug released is substantially uniform with minimal variance from stent to stent.

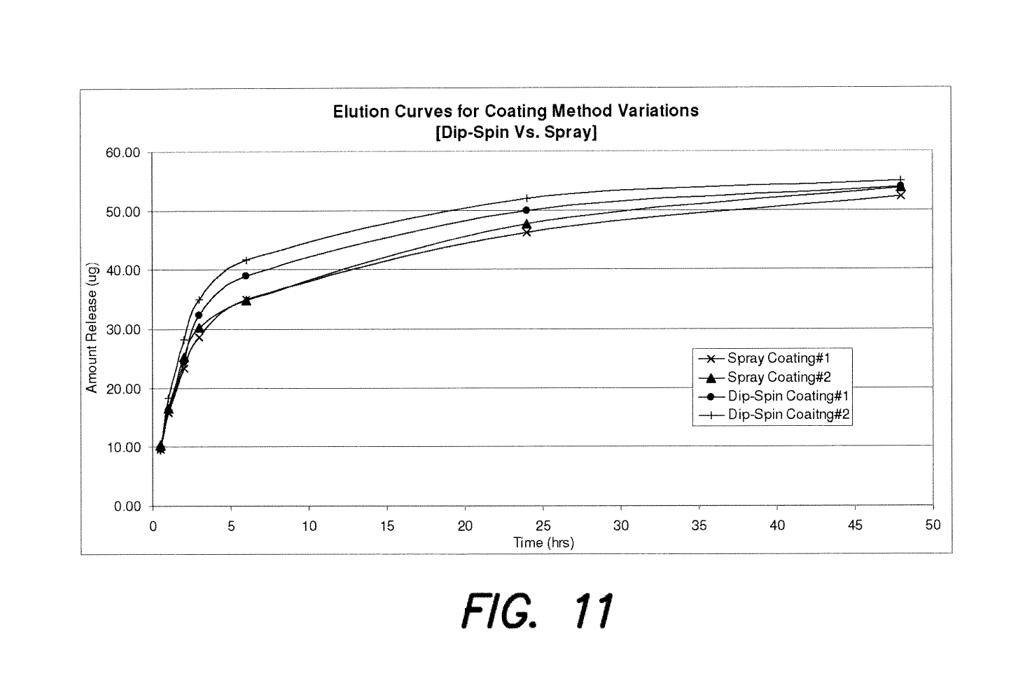

FIG. 11 is a graph resulting from an experiment similar to that of FIGS. 9 and 10, except two stents subjected to the multiple spray process of the present example are compared with two stents subjected to the dip/spin process of Example 3. It can be seen from FIG. 11 that despite the different processes used, the amount of drug released is substantially uniform from stent to stent.

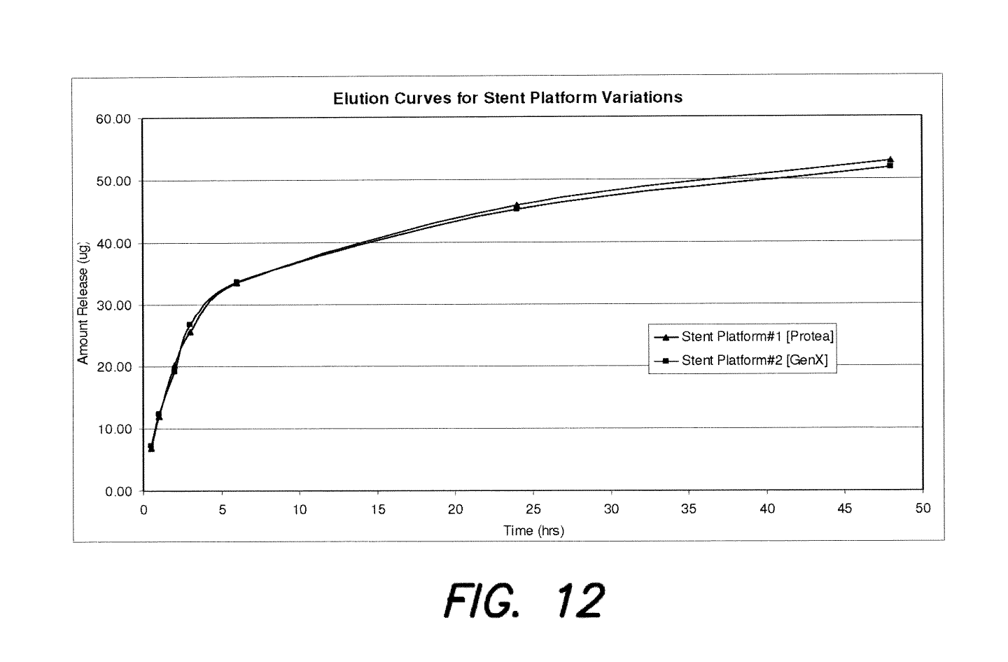

FIG. 12 is a graph resulting from an experiment similar to that of FIGS. 9-11, except that different stent designs are compared. A Protea™ stent (MIV Therapeutics, Inc.) and GenX™ stent (MIV Therapeutics, Inc.) are each coated by the method of Example 1 and then subjected to the multiple spray process of the present Example. Both stents have a similar surface area. FIG. 12 shows that the amount of drug eluted is substantially similar, despite the different stent designs.

1. A stent, comprising at least one coating covering at least a portion of the device, the at least one coating comprising:
a porous substrate having a thickness and an average pore diameter of less than 1 μm; and
a composition impregnating at least 50% of the thickness of the porous substrate, the composition comprising at least one lipid and at least one pharmaceutically effective agent.

2. The stent of claim 1, wherein the porous substrate has an average pore diameter ranging from 0.3 μm to 0.6 μm.

3. The stent of claim 1, wherein the porous substrate has a porosity volume ranging from 30 to 60%.

4. The stent of claim 1, wherein the porous substrate is coated on the stent.

5. The stent of claim 4, wherein the porous substrate has a thickness of 1 μm or less.

6. The stent of claim 5, wherein the porous substrate comprises a ceramic.

7. The stent of claim 6, wherein the ceramic is selected from metal oxides and calcium phosphates.

8. The stent of claim 7, wherein the ceramic is selected from hydroxyapatite.

9. A method of coating a stent, comprising:
(a) providing a stent, at least a portion of the stent having a porous substrate having an average pore diameter less than 1 μm; and
(b) spraying the stent with a fluid composition comprising at least one pharmaceutically active agent and at least one lipid to impregnate at least some of the pores of the porous substrate with the composition;
(c) spraying the stent with a solvent; and
(d) repeating steps (b) and (c) at least once.

10. The method of claim 9, wherein prior to the spraying in (b), the method further comprises spraying the porous substrate with a solvent.

11. The method of claim 9, wherein the repeating of (d) is performed at least twice.

12. The method of claim 9, wherein the porous substrate is the surface of the stent.

13. The method of claim 9, wherein the porous substrate is coated on at least a portion of the stent prior to step (a).

14. The method of claim 13, wherein the porous substrate comprises a ceramic.

15. The method of claim 14, wherein the porous substrate comprises a calcium phosphate.

16. The method claim 9, wherein the porous substrate has an average pore diameter ranging from 0.3 μm to 0.6 μm.

17. A method of coating a medical device comprising:
providing a medical device, at least a portion of the device having a porous substrate having an average pore diameter less than 1 μm; and
dipping the medical device in a composition comprising at least one lipid and at least one pharmaceutically active agent to impregnate at least some of the pores of the porous substrate with the composition
spinning the device to remove excess composition.

18. The method of claim 17, further comprising spraying the device with either a solvent or a dilute solution comprising the composition.

19. The method of claim 18, wherein the spinning occurs before the spraying.

20. The method of claim 18, wherein the spinning occurs simultaneously with the spraying.

21. The method of claim 17, wherein the providing comprises coating the porous substrate on at least a portion of the stent.

22. The method of claim 17, wherein the porous substrate has an average pore diameter ranging from 0.3 to 0.6 μm.

23. The method of claim 17, wherein the porous substrate has a porosity volume ranging from 30 to 60%.

24. The method of claim 17, wherein the porous substrate comprises a ceramic.

25. The method of claim 24, wherein the ceramic is selected from at least one metal oxide and at least one calcium phosphate.

26. The method of claim 25, wherein the at least one calcium phosphate is hydroxyapatite.

27. The method of claim 17, wherein the device is a stent, and the porous substrate has a thickness of no more than 2 μm.

28. The method of claim 17, wherein the device is a stent, and the porous substrate has a thickness of no more than 1 μm.

29. A method of coating a medical device comprising:
providing a medical device, at least a portion of the device having been previously coated with a porous substrate having an average pore diameter less than 1 μm;
subjecting the device to a vacuum; and
maintaining the vacuum while applying to the device a composition comprising at least one lipid and at least one pharmaceutically active agent to impregnate at least some of the pores of the porous substrate with the composition.

30. The method of claim 29, wherein the applying comprises spraying the device with the composition.

31. The method of claim 29, wherein the applying comprises dipping the device in the composition.

32. The method of claim 29, wherein the vacuum is at −20 mm Hg or greater.

33. A method of coating a medical device comprising:
providing a medical device, at least a portion of the device having been previously coated with a porous substrate having an average pore diameter less than 1 μm; and
dipping the medical device in a composition comprising at least one lipid and at least one pharmaceutically active agent to impregnate at least some of the pores of the porous substrate with the composition;
spraying the device with either a solvent or a dilute solution of the composition.

34. The method of claim 33, wherein after the dipping and/or spraying, the method further comprising spinning the device to remove excess composition.

35. The method of claim 34, wherein after the spinning, the method further comprises spraying the device with a solvent or a dilute form of the composition comprising the at least one pharmaceutically active agent.

36. The method of claim 33, wherein the exposing occurs under vacuum conditions.
